(19) United States
(12) Patent Application Publication
(10) Pub. No.: US 2015/0005390 A1
(43) Pub. Date: Jan. 1, 2015

(54) METHOD FOR TREATING INFLAMMATORY DISORDER

(71) Applicant: National Defense Medical Center, Taipei (TW)

(72) Inventors: Shuk-Man KA, Taipei (TW); Kuo-Feng HUA, I-Lan (TW); Ann CHEN, Taipei (TW)

(21) Appl. No.: 13/930,125

(22) Filed: Jun. 28, 2013

(51) Int. Cl. A61K 31/085

(52) U.S. Cl. CPC A61K 31/085
USPC 514/718

(57) ABSTRACT

The present invention relates to a method for treating inflammatory disorder, comprising administering to a subject in need thereof a therapeutically effective amount of a compound of 2-methoxy-4-methylphenol or a pharmaceutically acceptable salt or a physiologically functional derivative thereof, wherein the compound is in a therapeutically effective amount to inhibit NO or IL-6 expression, caspase-1 activation or IL-1β secretion, and NLRP3 and IL-1β precursor expression in inflammatory cells of the subject.

### FIELD OF THE INVENTION

The present invention relates to a new method for treating inflammatory disorders comprising administering to a subject in need thereof a therapeutically effective amount of creosol.

### BACKGROUND OF THE INVENTION

Creosol, also known as 2-methoxy-4-methylphenol, 2-methoxy-4-cresol or 4-methyl guaiacol, is a phenolic ingredient of creosote, which is commonly used as a disinfectant, wood preservative, antidiarrheic drug, or expectorant (Matsushima N et al., Eur J Pharmacol. 2007; 567 (1-2): 59-66). Creosol, which is found in wines that have matured in oak barrels, can also be prepared by hydrogenation of vanillin (Pérez-Prieto L J et al., J Agric Food Chem. 2003, 51 (18): 5444-9). Further, vanillin can be isolated from lignin in large scales through commercial processes (Meylemans H A et al., ChemSusChem. 2012, 5 (1): 206-10). Creosol, C₈H₁₀O₂, is a colorless liquid with a structure as below:

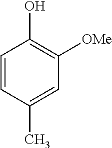

It was reported that creosol is able to prevent the cell death of cultured rat hippocampal neurons exposed to N-methyl-D-aspartate, or H₂O₂, by suppressing the Ca²⁺ influx and generating intracellular reactive oxygen species (Nakamichi N et al., J Neurosci Res. 2010, 88 (11): 2483-93). Creosol was also reported to be able to prevent ovariectomy-induced bone loss through inhibiting osteoclastogenesis, in association with an anti-oxidative property in osteoblasts (Moriguchi N et al., Biochem Pharmacol. 2007, 73 (3): 385-93).

Inflammation occurs in response to numerous conditions including physical injury, irritation, tumor growth in tissue, and bacterial, parasitic, fungal, or viral infection. Inflammation causes both local and systemic effects. Representative effects that can occur at the site of injury, irritation, or disease are the increase of vascular permeability, release of degradative enzymes including metalloproteinase, migration of leukocytes to the affected site, neutrophil burst response to destroy invading cells, and the secretion of cytokines. Important systemic effects include pain, fever, and the acute response in the liver.

A macrophage is a type of inflammatory cell that plays an important role in the inflammatory response. Once activated, macrophages can induce a series of inflammatory responses by releasing inflammatory mediators, against the infections or foreign particles. Further, nitric oxide (NO), interleukin-6 (IL-6), and TNF-α are important pro-inflammatory mediators that are produced mainly by lipopolysaccharide (LPS)-activated macrophages and mediate multiple biological effects, including the activation of immune responses.

Additionally, the inflammasome is a multi-protein signal complex for activating caspase-1. Among the inflammasome, the NLRP3 inflammasome is one of the most well-studied. The NLRP3 inflammasome is activated by adenosine triphosphate in LPS-activated macrophages, leading to caspase-1 activation and IL-1α secretion (Hu Y, et al., J Immunol. 2010 Dec. 15; 185 (12):7699-705).

Creosol has drawn the attention of medical researchers due to its ability to prevent cell death in hippocampal neurons and bone loss induced by ovariectomy. However, creosol has not been used to treat inflammation.

### BRIEF SUMMARY OF THE INVENTION

The present invention is based on the unexpected finding that 2-methoxy-4-methylphenol, creosol, may be used to treat inflammation. Therefore, the present invention provides a new approach for treatment of inflammation disorder.

In one aspect, the present invention provides a method for treating an inflammatory disorder, comprising administering to a subject in need thereof a therapeutically effective amount of a compound of formula (I):

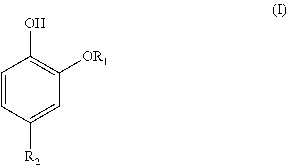

wherein each of R₁ and R₂, independently, is H or an alkyl.

In one embodiment of the present invention, the compound is 2-methoxy-4-methylphenol.

In another embodiment of the present invention, the compound is a therapeutically effective amount capable of inhibiting nitric oxide or interleukin-6 (IL-6) expression in inflammatory cells of the subject.

In a further embodiment of the present invention, the compound is a therapeutically effective amount capable of inhibiting caspase-1 activation or IL-1β secretion in inflammatory cells of the subject.

In yet another embodiment of the present invention, the compound is a therapeutically effective amount capable of inhibiting NLRP3 and IL-1β precursor expression in inflammatory cells of the subject.

In an additional embodiment of the present invention, the inflammatory cells are macrophages.

The details of one or more embodiments of the present invention are set forth in the description below. Other features or advantages of the present invention will be apparent from the following detailed description of several embodiments and from the appended claims.

### BRIEF DESCRIPTION OF THE DRAWINGS

For the purpose of illustrating the invention, there are shown in the drawings embodiments which are presently preferred. It should be understood, however, that the invention is not limited to the preferred embodiments shown. In the drawings:

FIGS. 1A to 1C show the effects of creosol on the expression of inflammatory mediators: NO (A), IL-6 (B), and TNF-α (C) (herein *p<0.05; **p<0.01).

FIG. 2A shows the effect of creosol on IL-1β (herein *p<0.05) when the cells were treated with creosol for 30 minutes, followed by LPS treatment for 6 hours; FIG. 2B shows the effect of creosol on caspase-1 (p45) and cleaved caspase-1 (p10) when the cells were treated with creosol for 30 minutes, followed by LPS treatment for 6 hours; FIG. 2C shows the effect of creosol on IL-1β (herein *p<0.05) when the cells were incubated with creosol for 30 minutes before LPS-priming; FIG. 2D shows the effect of creosol on caspase-1 (p45) and cleaved caspase-1 (p10) when the cells were incubated with creosol for 30 minutes before LPS-priming; and FIG. 2E shows the effect of creosol on NLRP3 inflammasomes and IL-1β precursor when the cells were incubated with creosol for 30 minutes, followed by LPS treatment for 6 hours.

### DETAILED DESCRIPTION OF THE INVENTION

Unless defined otherwise, all technical and scientific terms used herein have the meaning commonly understood by a person skilled in the art in the field of this invention. As used herein, the following terms have the meanings ascribed to them unless specified otherwise.

The articles “a” and “an” are used herein to refer to one or more (i.e., at least one) of the grammatical object of the article. For example, “an element” means one element or more elements.

The table below shows the abbreviations for some terminologies.

| | |
|---|---|
| NO | nitric oxide |
| IL-6 | interleukin-6 |
| TNF-α | tumor necrosis factor-α |
| IL-1β | interleukin-1β |
| LPS | lipopolysaccharide |
| ELISA | enzyme-linked immunosorbent assay |
| ATP | adenosine triphosphate |

In one aspect, the present invention is directed to a method for treating an inflammatory disorder, comprising administering to a subject in need thereof an effective amount of a compound of formula (I):

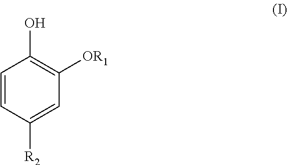

wherein each R₁ and R₂, independently, is H or an alkyl.

The term “alkyl” refers to a straight or branched monovalent hydrocarbon containing, unless otherwise stated, 1-20 carbon atoms (e.g., C₁-C₈). Examples of alkyl include, but are not limited to, methyl, ethyl, n-propyl, i-propyl, n-butyl, i-butyl, and t-butyl. Unless specifically pointed out, alkyl mentioned herein includes both substituted and unsubstituted moieties. The term “substituted” refers to one or more substituents each replacing a hydrogen atom.

The term “treating” as used herein includes prophylaxis of the specific disorder or condition or the alleviation of symptoms associated with a specific disorder or condition and/or eliminating said symptoms. For example, the term “treating an inflammatory disorder” as used herein will refer to reducing local or systemic inflammatory overresponses by inhibiting nitric oxide or IL-6 expression, inhibiting caspase-1 activation or IL-1β secretion, as well as inhibiting NLRP3 and IL-1β precursor expression in inflammatory cells of the subject.

The term “subject” as used herein includes human beings and animals, such as companion animals (e.g., dogs, cats, and the like), farm animals (e.g., cows, sheep, pigs, horses, and the like), or laboratory animals (e.g., rats, mice, guinea pigs, and the like).

The term “inflammatory disorder” as used herein includes rheumatoid arthritis, systemic lupus erythematosus, alopecia areata, ankylosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, juvenile dermatomyositis, discoid lupus, essential mixed cryoglobulinemia, fibromyalgia-fibromyositis, Grave's disease, Guillain-Barre, Hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

The term “therapeutically effective amount” as used herein refers to the amount necessary for each active agent to confer a therapeutic effect on the subject, either alone or in combination with one or more active agents. Effective amounts vary, as recognized by those skilled in the art, depending on route of administration, excipient usage, and co-usage with other active agents.

In the present invention, it was unexpectedly found that the compound of formula (I) would be useful in treating inflammation's effects, by reducing the expression of NO and IL-6 in LPS-activated macrophages, inhibiting NLRP3 inflammasomes mediated IL-1β expression and secretion by reducing capase-1 activation in ATP stimulated LPS-activated macrophages.

According to the present invention, the method for treating inflammatory disorder comprises administering to a subject in need thereof a therapeutically effective amount of the compound of formula (I), wherein each of R₁ and R₂, independently, is H or an alkyl.

In one embodiment, the compound of the present invention is an amount effective to inhibit NO or IL-6 secretion, caspase-1 activation, IL-1β secretion, and/or NLRP3 expression and IL-1β precursor expression in inflammatory cells, particularly in macrophages.

Without further elaboration, it is believed the above description has adequately enabled the present invention. The following example is, therefore, to be construed as merely illustrative, and does not limit of the remainder of the disclosure in any way whatsoever. All of the publications, including patents, cited herein are hereby incorporated by reference in their entireties.

### EXAMPLE

### 1. Materials

LPS (from Escherichia coli 0111:B4) and anti-actin antibodies were purchased from Sigma (St. Louis, Mo.). Anti-IL-1β and anti-caspase-1 antibodies were obtained from Santa Cruz Biotechnology (Santa Cruz, Calif.). TNF-α, IL-6, and IL-1β ELISA kits were purchased from R&D Systems (Minneapolis, Minn.). Anti-NLRP3 antibody was obtained from Enzo Life Science Inc. (Exeter, UK).

### 2. Cell Cultures

Murine macrophages RAW 264.7 and J774A.1 cells were obtained from the American Type Culture Collection (Rockville, Md.). RAW 264.7 macrophages stably transfected with the NF-κB reporter gene (RAW-Blue™ cells), purchased from InvivoGen (San Diego, Calif.). All cells were propagated in RPMI-1640 medium supplemented with 10% heat-inactivated fetal calf serum and 2 mM L-glutamine, and cultured at 37° C. in a 5% CO₂ incubator (RAW-Blue™ cells cultured in the presence of Zeocin™).

### 3. Enzyme-Linked Immunosorbent Assay (ELISA)

Cells were seeded in 6-well plates at a density of 5×10⁵ cells/ml, and then incubated with or without LPS (1 μug/ml) in the absence or presence of tested samples for 24 hours. The effects of the tested samples on TNF-α, IL-6 and IL-1β production were measured by ELISA according to the manufacturer's protocol. Briefly, 50 μl of biotinylated antibody reagent and 50 μl of supernatant were added to an anti-mouse TNF-α, IL-6 and IL-1β precoated stripwell plate, and incubated at room temperature for 2 hours. After washing the plate three times with the washing buffer, 100 μl of diluted streptavidin-HRP (horseradish peroxidase) concentrate was added to each well and the plate was incubated at room temperature for 30 minutes. The washing process was repeated; then, 100 μl of a premixed tetramethylbenzidine substrate solution was added to each well and developed at room temperature, in the dark, for 30 minutes. Following the addition of 100 μl of stop solution to each well to stop the reaction, the absorbance of the plate was measured by a microplate reader at a 450 nm wavelength.

### 4. NO Inhibitory Assay

RAW 264.7 cells were seeded in 24-well plates at a density of 5×10⁵ cells/ml, and then incubated with or without LPS (1 μg/ml) in the absence or presence of tested samples for 24 hours. The effects of creosol on NO production were measured indirectly by the analysis of nitrite levels, using the Griess reaction.

### 5. Western Blot Assay

Whole cell lysates were separated by SDS-PAGE and electrotransferred to a PVDF (polyvinylidene fluoride) membrane. The membranes were incubated in a blocking solution—5% nonfat milk in phosphate buffered saline with 0.1% Tween 20—at room temperature for 1 hour. Each membrane was incubated with a specific primary antibody, at room temperature, for 2 hours. After washing the membrane three times in PBS with 0.1% Tween 20, it was incubated with an HRP-conjugated secondary antibody directed against the primary antibody. The membrane was developed by an enhanced chemiluminescence Western blot detection system.

### 6. Statistical Analysis

All values are given as mean±SE. Data analysis involved one-way ANOVA with a subsequent Scheffe test.

### Results

### 1. Creosol Reduced NO and IL-6 in LPS-Activated Macrophages

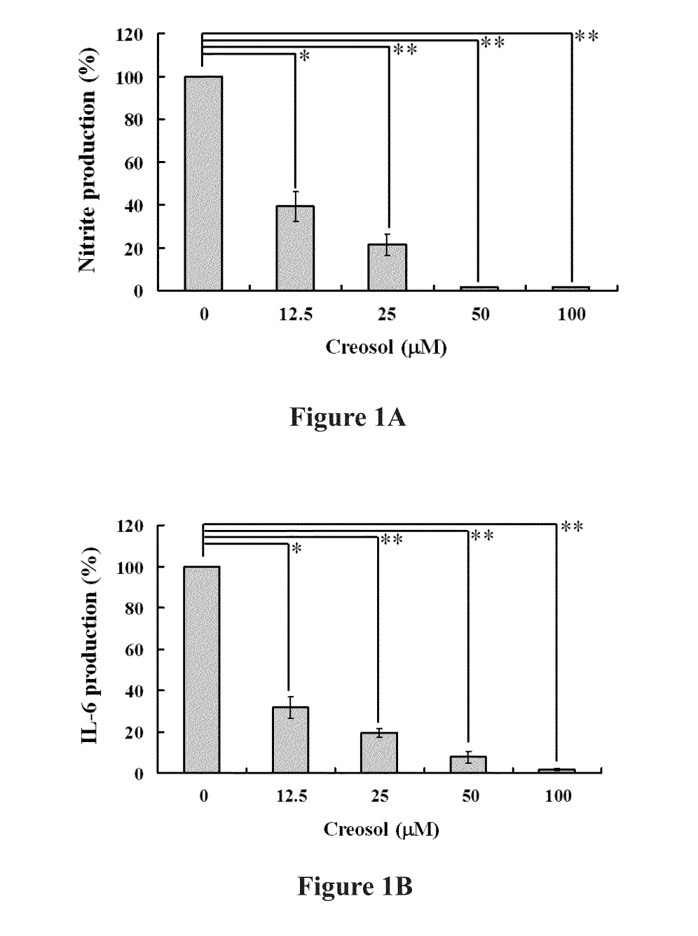

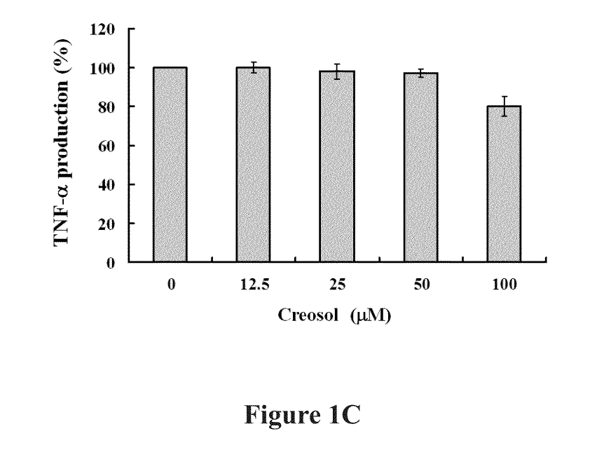

The effects of creosol on the expression of inflammatory mediators were analyzed. In the present invention, RAW 264.7 macrophages (5×10⁵/ml) were pretreated with creosol for 30 minutes, followed by stimulation with LPS (1 μg/ml) for 24 hours. NO concentration in culture medium was assayed by the Griess reaction. Data is expressed as a % of LPS alone ±SE from three separate experiments (wherein *p<0.05; **p<0.01), as shown in FIG. 1A.

As shown in FIG. 1B and FIG. 1C, RAW 264.7 macrophages (5×10⁵/ml) were pretreated with creosol for 30 minutes, followed by stimulating with LPS (1 μg/ml) for 24 hours. IL-6 and TNF-α concentration in culture medium was assayed by ELISA. Data is expressed as a % of LPS alone ±SE from three separate experiments (wherein *p<0.05; **p<0.01).

As mentioned above, NO, IL-6, and TNF-α are important pro-inflammatory mediators produced mainly by activated macrophages, and mediate multiple biological effects, including the activation of immune responses. In the present invention, the Griess reaction was used to characterize the NO expression dose-response in creosol-pretreated cells, and it was found that the expression of NO was inhibited in creosol-pretreated cells in a dose-dependent manner as shown in FIG. 1A. To evaluate the effect of creosol on cytokine expression in LPS-activated macrophages, an ELISA was conducted to characterize cytokine expression dose-response in creosol-pretreated cells. As shown in FIG. 1B, it was found that creosol was able to inhibit IL-6 expression in a dose-dependent manner; however, creosol was not able to inhibit TNF-α expression in LPS-activated macrophages as shown in FIG. 1C.

### 2. Creosol Reduced IL-1β Secretion Through Inhibiting NLRP3 Inflammasomes

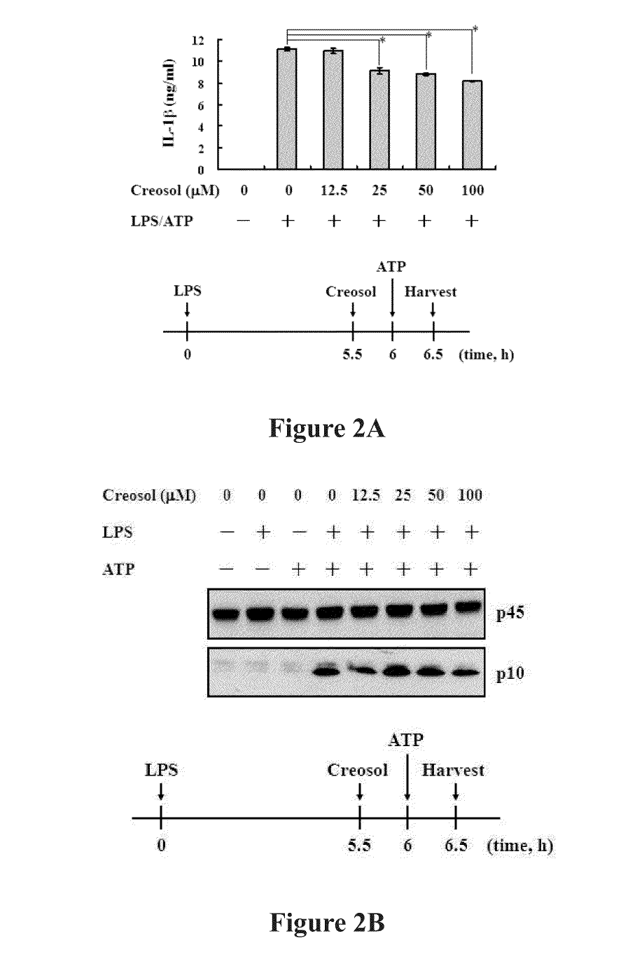

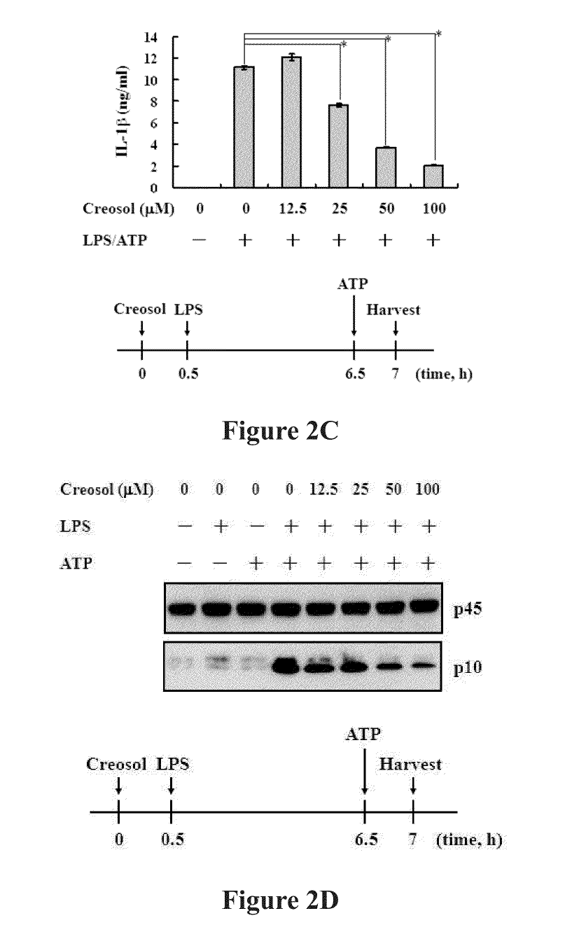

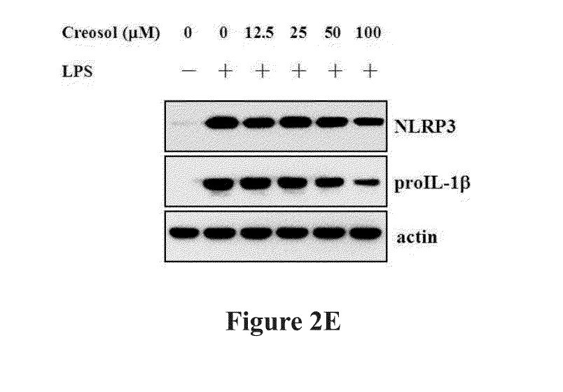

J774A.1 cells (1×10⁶/ml) were primed with LPS for 5.5 hours, followed by treatment with creosol for 30 minutes, and then stimulated with ATP (5 mM) for an additional 30 minutes. As shown in FIG. 2A, IL-1β concentration in culture medium was assayed by ELISA. Data is expressed as the mean±SE from three separate experiments (wherein *p<0.05). In FIG. 2B, the expression of caspase-1 (p45) and cleaved caspase-1 (p10) were analyzed by Western blot. The result of one of the three separate experiments is shown below. In the present invention, J774A.1 cells (1×10⁶/ml) were treated with creosol for 30 minutes, followed by LPS treatment for 6 hours. After the wash, cells were stimulated with ATP (5 mM) for an additional 30 minutes. As shown in FIG. 2C, IL-1β concentration in the culture medium was assayed by ELISA. Data is expressed as the mean±SE from three separate experiments (wherein *p<0.05). The expressions of caspase-1 (p45) and cleaved caspase-1 (p10) were analyzed by Western blot, and the result of one of the three separate experiments is shown in FIG. 2D. Further, J774A.1 cells (1×10⁶/ml) were treated with creosol for 30 minutes, followed by LPS treatment for additional 6 hours. The expressions of NLRP3 inflammasomes and IL-1β precursor were analyzed by Western blot. The result of one of the three separate experiments is shown in FIG. 2E.

ATP activates NLRP3 inflammasomes in LPS-primed macrophages, which leads to caspase-1 activation and IL-1β secretion. To test whether creosol is able to modulate NLRP3 inflammasomes activation, the mouse macrophage cell line, J774A.1, was selected, and the effect of creosol on NLRP3 inflammasomes activation was tested.

FIG. 2A shows the effect of creosol on IL-1β, and FIG. 2B shows the effect of creosol on caspase-1 (p45) and cleaved caspase-1 (p10) when the cells were treated with creosol for 30 minutes, followed by LPS treatment for 6 hours. Initially, NLRP3 inflammasomes activation in

J774A.1 cells treated with LPS-priming, and after ATP stimulation, was detected by measuring IL-1β secretion and caspase-1 activation. As shown in FIG. 2A, ATP induced IL-1β secretion in LPS-primed J774A.1 cells. To determine whether IL-1β secretion is impaired by creosol, LPS-primed J774A.1 cells were incubated with creosol for 30 minutes, and then ATP was added and incubated for another 30 minutes. In contrast to the vehicle control, creosol inhibited IL-1β secretion slightly. The effect of creosol on caspase-1 activation was also tested by immunoblotting the p10 subunit of mature caspase-1 in ATP stimulated LPS-priming J774A.1 cells. ATP induced caspase-1 activation in LPS-primed J774A.1 cells. To determine whether caspase-1 activation is impaired by creosol, LPS-primed J774A.1 cells were incubated with creosol for 30 minutes, before ATP was added, and incubated for another 30 minutes. Creosol inhibited caspase-1 activation slightly, as shown in FIG. 2B.

FIG. 2C shows the effect of creosol on IL-1β, and FIG. 2D shows the effect of creosol on caspase-1 (p45) and cleaved caspase-1 (p10) when the cells were incubated with creosol for 30 minutes, before LPS-priming. In addition, to know whether creosol inhibits NLRP3 inflammasomes activation through affecting LPS-mediated signaling, J774A.1 cells were incubated with creosol for 30 minutes, before LPS-priming. After LPS-priming, creosol and LPS were washed out, and ATP was added for another 30 minutes, before measuring IL-1β secretion and caspase-1 activation. As shown in FIG. 2C, creosol inhibited IL-1β secretion in a dose-dependent fashion. The caspase-1 activation is also inhibited by creosol in a dose-dependent fashion as shown in FIG. 2D.

FIG. 2E shows the effect of creosol on NLRP3 inflammasomes and IL-1β precursor. Furthermore, it was tested whether creosol could inhibit NLRP3 expression, an essential component of inflammasomes, as well as the IL-1β precursor in LPS-activated cells. Cells were incubated with creosol for 30 minutes, followed by LPS stimulation for another 6 hours. It was found that creosol inhibited NLRP3 expression slightly, but significantly inhibited the IL-1□ precursor expression in LPS-activated macrophages. These results indicate that creosol inhibited the NLRP3 inflammasomes activation through affecting LPS-mediated signaling.

Changes could be made, by those skilled in the art, to the embodiments described above without departing from the broad inventive concept thereof. Therefore, this invention is not limited to the particular embodiments disclosed, but it is intended to cover modifications within the spirit and scope of the present invention as defined by the appended claims.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

I/We claim:

1. A method for treating an inflammatory disorder, comprising administering to a subject in need thereof a therapeutically effective amount of a compound of formula (I): 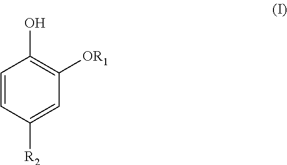
wherein each of R₁ and R₂, independently, is H or an alkyl.

2. The method according to claim 1, wherein the compound is 2-methoxy-4-methylphenol.

3. The method according to claim 1, wherein the compound is in a therapeutically effective amount to inhibit nitric oxide or IL-6 expression in inflammatory cells of the subject.

4. The method according to claim 3, wherein the inflammatory cells are macrophages.

5. The method according to claim 1, wherein the compound is in a therapeutically effective amount to inhibit caspase-1 activation or IL-1β secretion in inflammatory cells of the subject.

6. The method according to claim 5, wherein the inflammatory cells are macrophages.

7. The method according to claim 1, wherein the compound is in a therapeutically effective amount to inhibit NLRP3 and IL-1β precursor expression in inflammatory cells of the subject.

8. The method according to claim 7, wherein the inflammatory cells are macrophages.
